(19) United States
(12) Patent Application Publication
(10) Pub. No.: US 2007/0038090 A1
(43) Pub. Date: Feb. 15, 2007

(54) Ultrasound system for displaying an elastic image

(75) Inventors: Hae Yean Moon, Seoul (KR); Cheol An Kim, Seoul (KR); Ra Young Yoon, Seoul (KR)

Correspondence Address: C. IRVIN MCCLELLAND;OBLON, SPIVAK, MCCLELLAND, MAIER & NEUSTADT, P.C., 1940 DUKE STREET, ALEXANDRIA, VA, 22314, US

(73) Assignee: Medison Co., Ltd., Hongchun-gun (KR)

(21) Appl. No.: 11/492,780

(22) Filed: Jul. 26, 2006

(30) Foreign Application Priority Data
Jul. 27, 2005 (KR) 10-2005-0068269
Dec. 28, 2005 (KR) 10-2005-0131465

(51) Int. Cl. A61B 8/00

(52) U.S. Cl. 600/437; 600/438

(57) ABSTRACT

The present invention relates to an ultrasound system comprising: an ultrasound diagnosis unit for providing ultrasound scanning information of a target object and stress information applied to the target object, the ultrasound diagnosis unit including a probe having a stress applying unit and a stress measuring sensor around a scan plane thereof; an elastic image processor for forming an elastic image based on the ultrasound scanning information and the stress information; and a display unit for displaying the elastic image.

### FIELD OF THE INVENTION

The present invention generally relates to an ultrasound system, and more particularly to an ultrasound system for forming and displaying an elastic image that provides tissue information of a target object by measuring a modulus of tissue elasticity.

### BACKGROUND OF THE INVENTION

An ultrasound diagnostic system has become an important and popular diagnostic tool since it has a wide range of applications. Specifically, due to its non-invasive and non-destructive nature, the ultrasound diagnostic system has been extensively used in the medical profession. Modem high-performance ultrasound diagnostic systems and techniques are commonly used to produce two or three-dimensional diagnostic images of internal features of an object (e.g., human organs).

The ultrasound diagnostic system generally uses a wide bandwidth transducer to transmit and receive ultrasound signals. The ultrasound diagnostic system forms images of human internal tissues by electrically exciting an acoustic transducer element or an array of acoustic transducer elements to generate ultrasound signals that travel into the body. The ultrasound signals produce ultrasound echo signals since they are reflected from body tissues, which appear as discontinuities to the propagating ultrasound signals. Various ultrasound echo signals return to the transducer element and are converted into electrical signals, which are amplified and processed to produce ultrasound data for an image of the tissues. The ultrasound diagnostic system is very important in the medical field since it provides physicians with real-time and high-resolution images of human internal features without the need for invasive observation techniques such as surgery.

The ultrasound image is usually displayed in a Brightness-mode (B-mode) using reflectivity caused by an impedance difference between the tissues of the target object. However, if the reflectivity of the target object is hardly different from those of the neighboring tissues such as tumor, cancer or the like, then it is not easy to recognize the target object in the B-mode image. Further, an ultrasound elastic imaging technology has been developed to display an image of the target object by using mechanical characteristics of the target object. Such technology is very helpful for diagnosing lesions such as cancers.

The conventional elastic imaging technology displays the image of the target object by mapping relative strains of the tissues, which occur by applying stress to the target object, using pseudo colors. Such technology using pseudo colors is advantageous since it can easily display information relating to tissue hardness. However, there is a problem with the above technology in that the tissue hardness cannot be quantitatively displayed.

Also, it is difficult to clearly display the boundaries between the lesion and neighboring tissues with the conventional elastic imaging technology. Further, such technology is highly inconvenient since various operations must be carried out in order to measure information relating to radius and circumference of a tissue in an obtained image.

### SUMMARY OF THE INVENTION

The present invention provides an ultrasound system for forming an ultrasound image by measuring a modulus of elasticity of tissues and then forming and displaying an elastic image indicating the measured information of a target object.

According to an aspect of the present invention, there is provided an ultrasound diagnosis unit for providing ultrasound scanning information of a target object and stress information applied to the target object. The ultrasound diagnosis unit includes: a probe having a stress applying unit and a stress measuring sensor around a scan plane; an elastic image processor for forming an elastic image based on the ultrasound scanning information and the stress information; and a display unit for displaying the elastic image.

### BRIEF DESCRIPTION OF THE DRAWINGS

The above and other objects and features of the present invention will become apparent from the following descriptions of preferred embodiments given in conjunction with the accompanying drawings, in which:

FIG. 1 is a block diagram showing an ultrasound system constructed in accordance with the present invention;

FIG. 2 is a schematic diagram showing a spring model for explaining a modulus of elasticity;

FIG. 3 is a schematic diagram showing a structure of a probe constructed in accordance with the present invention;

FIG. 4 is a diagram showing an example of a selection window;

FIG. 5A is a photograph showing an elastic image before an image enhancing process is performed;

FIG. 5B is a photograph showing an elastic image in which an image enhancing process is performed; and

FIG. 6 is a photograph showing an elastic image performed in an automatic measure mode in accordance with the present invention.

### DETAILED DESCRIPTION OF THE PRESENT INVENTION

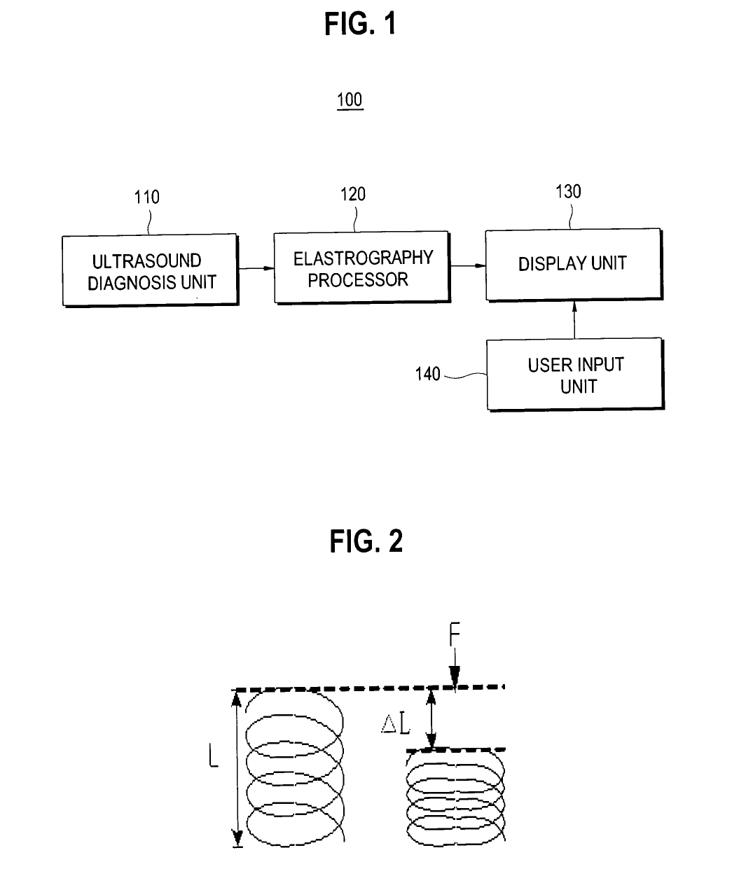

FIG. 1 is a block diagram showing an ultrasound system constructed in accordance with the preferred embodiment of the present invention. As shown in FIG. 1, the ultrasound system 100 includes an ultrasound diagnosis unit 110, an elastrography processor 120, a display unit 130 and a user input unit 140. The ultrasound diagnosis unit 110 includes a probe having a stress applying unit around a scan surface thereof and a stress measuring sensor positioned on the stress applying unit. The ultrasound diagnosis unit 100 provides ultrasound scanning information of the target object and strength information of the stress applied to the target object. The target object may be a reflector included in a subject. Further, the probe may be a 3-dimensional probe.

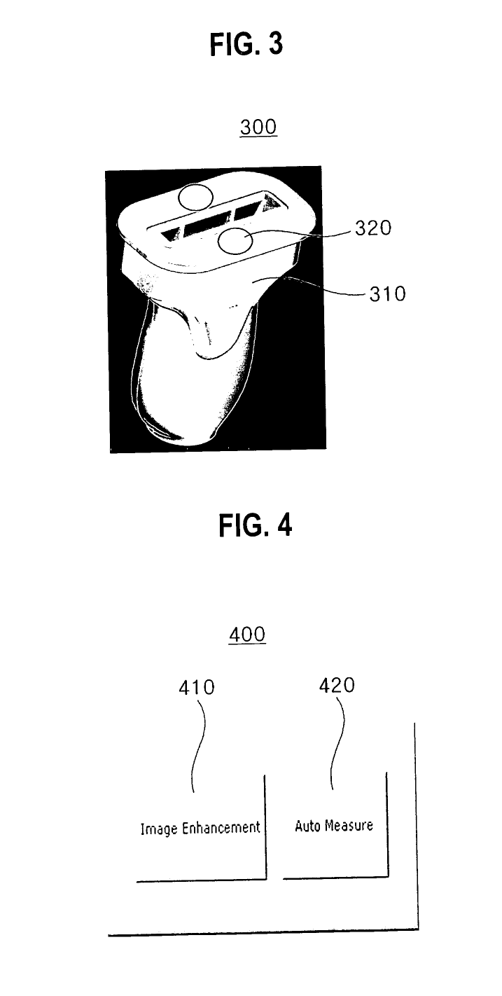

The information relating to the stress applied to the tissues should be obtained in order to accurately form an elastic image and to quantitatively measure the modulus of elasticity of the tissues. In order to deeply and uniformly apply the stress to the target object along a vertical direction, a probe, the scan surface of which is provided with a stress applying unit 310, is employed (shown in FIG. 3). The stress applying unit 310 has an opening exposing the scan surface of the probe at a center thereof. The contact surface of the stress applying unit 310 should be greater than the scan surface of the probe so that the stress can be more deeply and uniformly applied to the tissues within the human body. A stress measuring sensor 320 may be attached to the contact surface of the stress applying unit 310. The strength information of the stress measured by the stress measuring sensor 320 is transferred to the elastic image processor 120. The stress measuring sensor 320 may be a cable-type or wireless-type sensor.

The elastic image processor 120 forms an elastic image based on the ultrasound scanning information and the strength information of the stress. If the stress measuring sensor 320 is a cable-type sensor, then the strength information of the stress is directly transmitted from the stress measuring sensor 320 to the elastic image processor 120. However, if the stress measuring sensor 320 is a wireless-type sensor, then the ultrasound system 100 may further include a wireless signal receiving unit and a wireless signal processing unit (not shown). The strength information of the stress is transmitted to the elastic image processor 120 through the wireless signal processing unit.

The modulus of elasticity may be obtained by measuring the strain obtained by comparing the ultrasound signals reflected from the target object before and after the stress is applied. The modulus of elasticity may be defined by using the 1-dimensional spring model illustrated in FIG. 2. A force F required for compressing the spring by a predetermined depth is proportional to the modulus of elasticity. That is, if the stress applied to a unit area is a and the strain is ε, then the modulus of elasticity (E) is defined as the following equation:

$$E = \frac{σ}{ɛ} ( σ = F / A , ɛ = Δ L / L ) \qquad ( 1 )$$

Wherein “A” represents an area to which the stress is applied, “L” represents a length of the spring not applying the stress, and “ΔL” represents a length variation of the spring according to the stress applied. The modulus of elasticity is referred to as the Young's modulus. The modulus of elasticity of the target object is measured by calculating the ratio of a length not subjected the stress to a length subjected to the stress. However, since the degree of the strain of the tissues in the human body cannot be directly measured, the strain is usually measured with the assumption that the stress is uniformly distributed in the human body. The elastic image is formed based on the measured strain.

The user input unit 140 receives an input from a user for selecting an image enhancement mode or an automatic measure mode for the elastic image. A selection window can be displayed on a screen of the display unit 130 displaying the current elastic image for mode selection (shown in FIG. 4). The selection widow provides a first button 410 for selecting an image enhancement mode and a second button 420 for selecting an automatic measure mode. If the image enhancement mode is selected by the user, then the image processor 120 performs image processing to clearly separate the boundaries between the lesions and neighboring tissues.

Hereinafter, an image enhancing method, which is performed in the image processor 120, will be described. If the selection information for selecting the image enhancement mode is inputted through the user input unit 140, then a mask of M×N size is applied to an elastic image currently displayed on a screen, wherein M and N are positive integers. Then, a threshold value is set based on the color values of entire pixels in the elastic image. Subsequently, a sum of the differences of the color values between neighboring pixels, which exist in the M×N mask, is calculated and the calculated sum is compared with the threshold value. If the sum is smaller than the threshold value, then the elastic image is smoothened by using a convolution mask, which is generally used to blur the image. According to the above process, normal tissues are displayed in a relatively smooth image in comparison to the lesion tissues.

If the sum is greater than the threshold value, a sharpening process is carried out for the elastic image. The sharpening process is carried out by applying a high-pass filter to the M×N mask. Since color differences between the lesion tissues are greater than those between the normal tissues, the boundaries between the lesion tissues and the normal tissues can be further sharpened through the sharpening process.

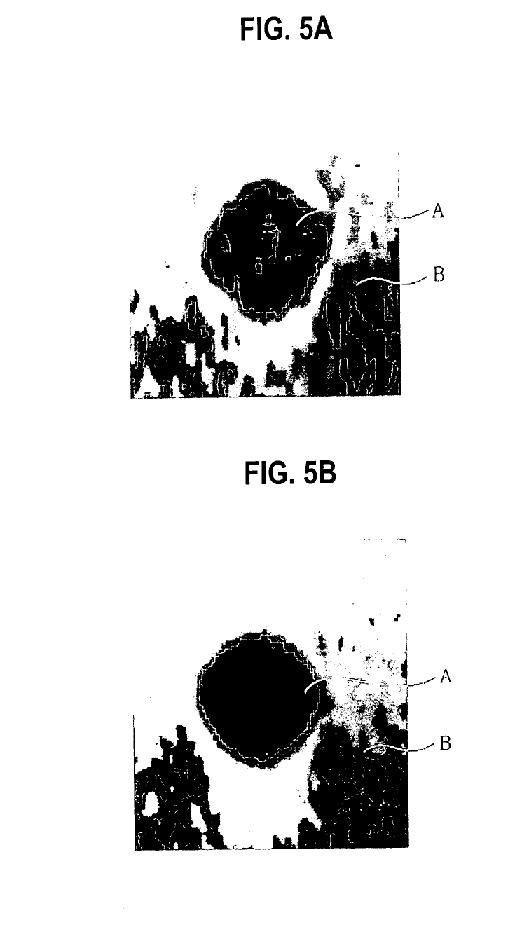

FIG. 5A is a photograph showing an elastic image before the image enhancement process is performed. FIG. 5B is a photograph showing an elastic image after performing the image enhancement process of the present invention. As shown in FIGS. 5A and 5B, an enhanced elastic image can be obtained through the image enhancement process of the present invention. Symbols A and B in FIGS. 5A and 5B represent the lesion and normal tissues (adjacent to the lesion), respectively.

Hereinafter, the operation of the automatic measure mode will be described. If the automatic measure mode is selected by the user through the selection window shown in FIG. 4, then a user interface such as a mouse pointer is activated on the elastic image. A specific lesion is selected by the user through the user interface and then the user interface is inactivated. After the lesion is selected, an automatic measure function used in a conventional ultrasound system is executed. This is so that the boundaries of the selected lesion are extracted and the diameter of the lesion is indicated based on the boundary information of extracted lesion. Different color information between the normal tissues and lesion tissues is used to trace the normal tissues and the lesion tissues. Trace coordinates (horizontal and vertical) of the boundaries between the normal tissues and the lesion tissues are stored. The difference between the minimum coordinate value and the maximum coordinate value is used to calculate the diameter of the lesion.

Further, an area of the lesion is calculated by using the stored trace coordinates and the coordinates of a mouse point selecting the lesion through the user interface. The areas of triangles drawn with the coordinates of the mouse point and the two traced coordinates are calculated. Then, the calculated areas are summed to calculate an area of the lesion. In order to calculate the area of the triangle, one of the trace coordinates is selected as first trace coordinates and a threshold is set to select the second trace coordinates around the first trace coordinates. If the area of the lesion is calculated by using all trace coordinates, then errors occurring in the area calculation of the triangles can be accumulated. Therefore, the threshold is set to minimize the errors.

The second trace coordinates are selected in a predetermined direction by using the threshold. The second trace coordinates correspond to coordinates, which render the distance difference between the first trace coordinates and the neighboring trace coordinates to be most approximate to the threshold. A first area of a first triangle drawn by the first trace coordinates, the second trace coordinates and the coordinates of the mouse point is calculated. Subsequently, third trace coordinates are selected by using the threshold. Further, a second area of a second triangle drawn by the second trace coordinates, the third trace coordinates and the coordinates of the mouse point is calculated. A fourth, fifth . . . n^{th} trace coordinates are selected and the areas of the triangles are calculated as in the above process. The first trace coordinates are selected to calculate an area of a last triangle regardless of the threshold. That is, the last triangle is drawn by the nth trace coordinates, the first trace coordinates and the coordinates of the mouse point. The calculated diameter and the area of the lesion are indicated on the elastic image.

Also, if the user selects the automatic measure mode, then a volume of lesion can be measured in a 3-dimensional space by using 3-dimensional data obtained by using the 3-dimensional probe. The boundaries between the lesion and the normal tissues can be detected in the same manner as mentioned above. If the lesion is a sphere, then the volume of the lesion can be calculated by the equation of 4/3πr³. However, since the lesion cannot be considered as a sphere, the volume of the lesion is calculated by measuring the diameters in axial, lateral and elevational directions. If the diameters in the axial, lateral and elevational directions are x, y and z, respectively, then the volume of the lesion can be approximately calculated by using the equation of 4/3πxyz. The calculated volume is displayed on the elastic image display unit.

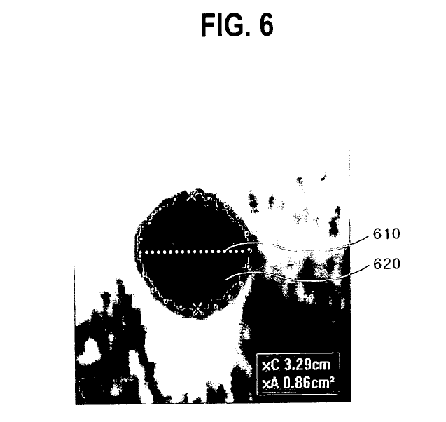

FIG. 6 is a photograph showing an elastic image performed in the automatic measure mode of the present invention. If the user selects a lesion on the elastic image displayed on the display unit and the automatic measure mode in the selection window shown in FIG. 3, then a diameter 610 and an area 620 of the selected lesion are automatically measured and displayed on the display unit (shown in FIG. 6).

As mentioned above, since the modulus of the elasticity is quantitatively calculated and displayed in a numerical value on the elastic image display unit, the hardness of the lesion can be perceived intuitively. Therefore, the lesion can be easily diagnosed.

Also, the present invention provides the enhancement mode and the automatic measure mode of the elastic image so that a desirable elastic image can be provided. Further, since the diameter and the area of the lesion can be automatically measured and the volume of the lesion in the 3-dimensional space can be measured by using the volume data, the characteristics of the lesion can be rapidly and conveniently diagnosed.

While the present invention has been described and illustrated with respect to a preferred embodiment of the invention, it will be apparent to those skilled in the art that variations and modifications are possible without deviating from the broad principles and teachings of the present invention, which should be limited solely by the scope of the claims appended hereto.

What is claimed is:

1. An ultrasound system, comprising:
an ultrasound diagnosis unit for providing ultrasound scanning information of a target object and stress information applied to the target object, the ultrasound diagnosis unit including a probe having a stress applying unit and a stress measuring sensor around a scan plane thereof;
an elastic image processor for forming an elastic image based on the ultrasound scanning information and the stress information; and
a display unit for displaying the elastic image.

2. The ultrasound system of claim 1, wherein the elastic image processor calculates a strain of the target object based on ultrasound receiving signals obtained before and after applying the stress, wherein the elastic image processor calculates a modulus of elasticity of the target object based on the stress information and the strain, and wherein the elastic image processor forms the elastic image based on the ultrasound scanning information and the modulus of elasticity of the target object.

3. The ultrasound system of claim 2, further comprising a user input unit for receiving an input from a user to select one of an image enhancement mode and an automatic measuring mode.

4. The ultrasound system of claim 3, wherein the elastic image processor enhances the elastic image displayed on the display unit in response to selecting the image enhancement mode, and wherein the display unit displays the enhanced elastic image.

5. The ultrasound system of claim 3, wherein the elastic image processor generates automatic measure information of a selected region from the target object in response to selecting the automatic measure mode, and wherein the display unit displays the automatic measure information.

6. The ultrasound system of claim 5, wherein the automatic measure mode is at lease one of a diameter, an area and a volume of the selected region.
